(19) United States
(12) Patent Application Publication
(10) Pub. No.: US 2010/0055079 A1
(43) Pub. Date: Mar. 4, 2010

(54) Method of Harvesting, Isolating, and Culturing Neural Stem Cells and Related Methods of Treating a Patient

(76) Inventor: Raymond F. Sekula, JR., Pittsburgh, PA (US)

Correspondence Address: ECKERT SEAMANS CHERIN & MELLOTT, 600 GRANT STREET, 44TH FLOOR, PITTSBURGH, PA, 15219, US

(21) Appl. No.: 12/572,781

(22) Filed: Oct. 2, 2009

Related U.S. Application Data
(63) Continuation-in-part of application No. 12/038,851, filed on Feb. 28, 2008.

(51) Int. Cl. A61K 35/12; C12N 5/02

(52) U.S. Cl. 424/93.7; 435/377

(57) ABSTRACT

The present invention provides a method of producing purified neural stem cells, comprising harvesting fluid containing neural stem cells from cerebrospinal fluid surrounding the spinal cord of an individual, isolating the neural stem cells from the fluid, culturing the neural stem cells in a culture medium effective to induce proliferation of the neural stem cells and purifying the cultured neural stem cells. Also provided is a method of treating a patient afflicted with a neurological condition, in which the purified neural stem cells are administered autologously into the same individual or heterologously to a patient other than the individual. Administration of the purified neural stem cells results in the purified neural stem cells propagating in the site of the brain region afflicted with the neurological condition.

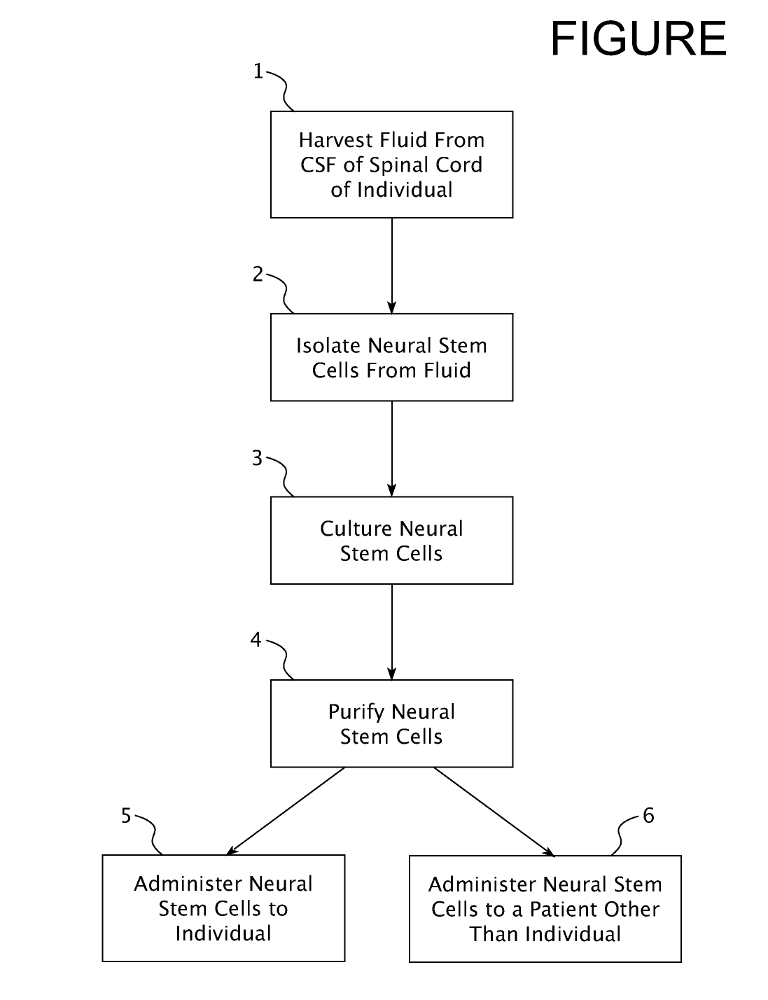

### CROSS-REFERENCE TO RELATED APPLICATIONS

The present application is a continuation-in-part of U.S. patent application Ser. No. 12/038,851, filed Feb. 28, 2008, and entitled “A Method of Producing Purified Neural Stem Cells and Related Methods of Treating a Patient,” which claims the benefit of U.S. Provisional Patent Application Ser. No. 60/893,780, filed Mar. 8, 2007, and entitled “Method of Producing Purified Neural Stem Cells and Related Methods of Treating a Patient.”

### BACKGROUND OF THE INVENTION

1. Field of the Invention

The present invention relates to the harvesting, isolation, culturing, purification, and propagation of neural stem cells, and more particularly, to the harvesting, isolating, and culturing of neural stem cells from the cerebrospinal fluid surrounding spinal cord or spinal nerve roots in order to administer and propagate neural stem cells in patients in need thereof.

2. Description of the Prior Art

Neurological conditions affect a large segment of the human population. With the percentage of people entering their elder years expected to increase in the next several decades, the percentage of people afflicted with a neurological condition undoubtedly is expected to increase as well. One of the most prevalent neurological conditions is stroke, which is the leading cause of disability worldwide, and is the third leading cause of death and disability in the United States (Kondziolka, D. et al., J. Neurosurg., 103:38-45, 2005). Beyond rehabilitation therapy following a stroke, once recovery from the stroke has reached a plateau and the neurological deficits are fixed, there are no accepted treatments to improve these neurological deficits.

Although cellular therapy for stroke is in its infancy, promising research in this area has been conducted. Phase I (Kondziolka, D. et al., Neurology, 55:565-569, 2000) and Phase II (Kondziolka, D. et al., J. Neurosurg., 103:38-45, 2005) trials of xenotransplanted neuronal cells derived from a human teratocarcinoma cell line have demonstrated safety and feasibility in human volunteers. These human subjects, however, required long-term immunosuppression.

Central nervous system (“CNS”) diseases and disorders are major health issues. Diseases and disorders of the CNS account for more hospitalizations, more long-term care, and more chronic suffering than nearly all other disorders combined. CNS diseases and disorders represent the largest and fastest growing area of unmet medical need. They generate more in total direct (healthcare related) and indirect (income) cost than any other therapeutic area: an estimated $1 trillion annually worldwide and over $350 billion annually in the U.S. The major classes of CNS diseases and disorders include neurodegenerative diseases, psychological and behavioral disorders, stroke, pain, cancer, epilepsy, traumatic brain injury (“TBI”), and spinal cord injury (“SCI”).

Adult human neural stem cells present therapeutic opportunities in cellular transplantation for SCI. Although human cellular therapy for SCI is in its infancy, promising research involving the transplantation of human cells into the damaged brain or spinal cord has been conducted. Advances in stem cell biology have raised expectations that grafts with the potential to differentiate into all major cell types of the spinal cord will be able to replace neurons and glial cells destroyed or rendered dysfunctional by injury and will ultimately become the ideal cells for regenerative medicine in CNS disorders.

The precedent for such intervention in human patients has been established in Parkinson's disease, where the transplantation of human fetal dopaminergic neurons has proven to be safe, clinically effective, and persistent, with allografts surviving for up to three years. While early results were encouraging, the limited availability of fetal tissue, coupled with the moral and ethical objections to its use, precluded widespread clinical application. The positive results from these studies, however, have motivated the search for another source of tissue.

Post-mitotic neurons derived from a human teratocarcinoma cell line have been transplanted into patients with strokes. Phase I and Phase II trials of these xenotransplanted neuronal cells have demonstrated safety and feasibility in human volunteers. Evidence for host integration of the transplanted grafts is lacking and graft survival following the withdrawal of immunosuppressants is uniformly poor.

Researchers from Sweden recently observed that strokes in rats cause the brain's own stem cells to divide and give rise to new neurons. The number of endogenous neural stem cells, however, activated following stroke is small in comparison to the number lost with injury, and much work lies ahead.

In Batten's disease, a rare and fatal neurodegenerative condition afflicting infants, a Phase I trial began in November, 2006 investigating the use of fetal-derived human neural stems transplanted into the brains of the patients in the hopes of producing the missing enzymes. The primary objective of the trial will be to measure the safety of transplanted human fetal neural stem cells (HuCNS-SC). Results of the Phase I trial are also expected to provide pre-clinical data that transplantation with human neural stem cells may lead to a possible treatment for NCL.

Today, much of the work involving neural stem cells (“NSCs”) in cellular transplantation for neurological disorders involves allogeneic and xenogeneic sources. In the past few years, however, several groups have isolated NSCs from the subventricular zone (“SVZ”), parenchyma, and dentate gyms of fetal, and adult human brains undergoing neurosurgical procedures and techniques for characterizing neural stem cells have been established. These studies have opened a possible scenario of autotransplantation, whereby NSCs are harvested from a patient, maintained, and expanded in vitro, induced to differentiate into all three neural cells types (neurons, astrocytes, oligodendrocytes), enhanced for certain genotypic or phenotypic properties, and selectively transplanted back into the patient. In this scenario, concerns of tumorigenicity or immunorejection are avoided, and most likely, an improved host response will be realized.

In a preliminary step toward autotransplantation, Olstorn et al. published the results of an experiment, whereby adult human neural stem cells (obtained from patients undergoing temporal lobectomy for intractable epilepsy) were transplanted into a rat model of transient global ischemia, survival and targeted migration into the lesioned CA1 region are shown, as well as differentiation into both a glial and an immature neuronal phenotypes. No signs of tumor formation or aberrant cell morphology were observed.

Still, the promise of autologous NSCs is tempered by certain limitations: detailed methods for harvesting neural stem cells from the brain are unavailable, and once isolated, only minute quantities are available. In addition, expansion of the harvested tissue into sufficient and relevant numbers for therapeutic intervention is required. Given the potential of stem cells and the priorities for clinical application, there is an urgent need to understand the promises and pitfalls of this unique approach to cell replacement and to apply it for effective treatments of SCI and other neurological disorders. There is also an urgent need to find reliable and safe sources for human neural stem cells with a potential for autologous grafting.

Because stem cell transplants are routinely used to treat patients with cancers and other disorders of the blood and the immune system, the recent identification of neural stem cells in the human nervous system may provide a platform for cellular-based therapies for a variety of neurological conditions, including traumatic brain injury, traumatic spinal cord injury, stroke, Parkinson's Disease, multiple sclerosis, and others.

In embryonic neurogenesis, the proliferation of neuronal precursors takes place at the surface of the central canal lining of the neural tube. The central ultimately forms the ventricular system of the adult. This neurogenic layer of thick, pseudostratified, columnar neuroepithelium is referred to as the ventricular/subventricular zone in adults. In development, mitogenesis in the ventricular/subventricular zone is followed by the migration of newly-generated neurons and glia along radial guide fibers into the brain parenchyma, including that of the cortical plate.

During the twenty-five years after the initial studies presented by Altman, Reynolds, and Weiss made the landmark discovery that neural stem cells could be isolated from the adult mouse brain and maintained in culture via propagation of floating cell clusters termed “neurospheres,” additional studies were made. These cells were soon isolated from the subependymal lining of the ventricular system throughout the mammalian species and determined to be the source of newly-recruited neurons in the rodent olfactory bulb. More recently, neural stem cells have been harvested from the hippocampi and subventricular zone (“SVZ”) adjacent to the wall of the lateral ventricle in the brains of adult human subjects undergoing neurosurgical procedures. Stem cells can be operationally defined as cells with the capacity to divide, self-renew, and differentiate into mature cell types. Multipotent neural stem cells (“NSC”) have the potential to generate the three major cell types in the CNS including neurons, astrocytes, and oligodendrocytes, while lineage-restricted neural precursor cells (“NPC”) have a more limited self-renewal and differentiation potential and are typically committed to either neuronal or glial fates. During development, cellular differentiation in the mammalian CNS occurs through sequential stages of lineage-restriction of NPC in a highly-specified and regulated manner by well-balanced spatial and temporal cues in the environment and intrinsic determinants within the cells.

In the past few years, several groups have isolated NSCs from the subventricular zone (“SVZ”) and hippocampi of fetal and adult human brains undergoing neurosurgical procedures. These studies have opened a possible scenario of autotransplantation, whereby NSCs are harvested from a patient, maintained and expanded in vitro, induced to differentiate into all three neural cells types (neurons, astrocytes, oligodendrocytes), enhanced for certain genotypic or phenotypic properties, expanded to clinically relevant volumes, and selectively transplanted back into the patient. In this scenario, concerns of tumorigenicity or immunorejection are avoided, and most likely, an improved host response will be realized. Unfortunately, harvesting of stem cells directly from the brain and spinal carries a high risk of morbidity and even mortality and will likely preclude this method of routine harvestation in the near future.

Several groups of investigators recently have isolated neural stem cells from both fetal and adult human brains (Arsenijevic, Y. et al., Exp. Neurol., 170:48-62, 2001; Johansson, C. B. et al., Exp. Cell Res., 253:733-736, 1999). For example, neural stem cells recently have been harvested from the subventricular zone (“SVZ”) adjacent to the wall of the lateral ventricle in the brains of adult human subjects undergoing neurosurgical procedures (Moe, M. C. et al., Neurogsurgery, 56:1182-1188, discussion 1188-1190, 2005; Sanai, N. et al., Nature, 427:740-744, 2004; Westerlund, U. et al., Neurosurgery, 57:779-784, discussion 779-784, 2005).

Neural stem cells proliferate during development of the central nervous system, giving rise to transiently dividing progenitor cells that eventually differentiate into the cell types that compose the adult brain. Stem cells generally have been defined as being capable of self-renewal, proliferation, and differentiation into multiple different phenotypic lineages. Specifically, with respect to neural stem cells, this includes neurons, astrocytes, and oligodendrocytes.

The discovery of neural stem cells in the adult brain and the feasibility of neuronal transplantation presents the possibility of autotransplantation, where neural stem cells are harvested from an individual and propagated and developed in vitro before they are used as transplants.

Ways of isolating, culturing and differentiating neural stem cells obtained from the central nervous system of animals and humans are known in the art. See, for example, U.S. Pat. No. 6,767,738, U.S. Pat. No. 6,777,233 and U.S. Pat. No. 6,897,060. Neural stem cells also have been shown to populate the cerebrospinal fluid of preterm patients with posthemorrhasic hydrocephalus (Krueger, R. C. et al., J. Pediatr., 148:337-340, 2006). In this study, Krueger et al. evaluated cerebrospinal fluid (CSF) from premature infants with posthemorrhasic hydrocephalus for the presence of neural progenitors. Over 95% of the CSF was obtained by an indwelling ventricular reservoir in the brain and the remainder from lumbar puncture. Regardless of what method was used, neuroprogenitor cells could be cultured from nearly all samples taken from premature infants with hydrocephalus. No cells were cultured from the CSF obtained by lumbar puncture from control premature infants. The logical conclusion from this paper is that if the individual source, i.e., premature infants, did not have posthemorrhasic hydrocephalus, then no cells in the CSF would be found.

There remains a need, therefore, for an efficient, cost-effective way to harvest neural stem cells from an individual which does not pose a risk of injury and death to the individual.

### SUMMARY OF THE INVENTION

The present invention relates to a method or technique of isolating central nervous system stem cells from a source from which they have never before been isolated.

The present invention meets the above need by providing a method of producing purified neural stem cells. The method comprises harvesting fluid containing neural stem cells from cerebrospinal fluid surrounding the spinal cord or spinal nerve roots of an individual, isolating the neural stem cells from the fluid, culturing the neural stem cells in a culture medium effective to induce proliferation of the neural stem cells, and purifying the cultured neural stem cells.

The present invention is directed to neural stem cells isolated from cerebrospinal fluid along the spinal axis that are cultured and filtered, and the therapeutic uses of such stem upon thawing. Such cells can be therapeutically valuable for reconstitution of the central nervous system in patients with various diseases and disorders. In a preferred embodiment, neural stem that have been cryopreserved and thawed can be used for autologous transplantation.

The harvesting of the fluid is effected by intrathecal aspiration of the cerebrospinal fluid (“CSF”) contained in the annular region surrounding the spinal cord of an individual. The regions of the spinal cord from which CSF is aspirated include the cervical region down to the sacral region and all regions in between. Preferably, CSF is aspirated from the fluid surrounding the lumbar region. Fluid from the CSF is collected in a syringe having a needle ranging in length of between 1 inch to 6 inches, preferably 3.5 inches. The gage of the needle can range from 10-27 gage, preferably 18-22 gage.

The amount aspirated from the CSF surrounding the spinal cord of the patient ranges from between about 5 ml to about 20 ml.

Imaging techniques, such as fluoroscopic imaging, may be used to ensure that the needle is placed in the correct location of the annular region of the site of aspiration.

The present invention also provides a method of treating a patient afflicted with a neurological condition. The method comprises harvesting fluid containing neural stem cells from cerebrospinal fluid surrounding the spinal cord of an individual, isolating the neural stem cells from the fluid, culturing the neural stem cells in a culture medium effective to induce proliferation of the neural stem cells, purifying the cultured neural stem cells, and administering the purified neural stem cells into a patient in need thereof. The neural stem cells harvested from an individual may be administered autologously to the same individual or may be administered heterologously to a patient other than the individual.

Administration of the purified neural stem cells results in the purified neural stem cells propagating in or adjacent to the site of the brain region afflicted with the neurological condition. For example, in the case of a stroke victim, the purified neural stem cells may be administered in the region of the brain where the stroke occurred. In addition, the purified stem cells may be administered in a region remote from the site of the brain region afflicted with the neurological condition, such as a vein, as the neural stem cells are capable of delivering themselves to the afflicted site and seed themselves therein.

Neurological conditions that can be treated according to the methods of the present invention include neurodegenerative or neurological diseases such as, for example, stroke, traumatic brain injury, traumatic spinal cord injury, Parkinson's disease, Alzheimer's disease, Huntington's disease, multiple sclerosis, or depression.

Techniques of purifying the neural stem cells are well known in the art and include, without limitation, immunocytochemical purification.

It is an object of the present invention to provide novel human central nervous system stem cells.

It is a further object of the present invention to provide an improved method of harvesting neural stem cells in order to isolate, culture and purify the neural stem cells to provide a source of neural stem cell for patients in need thereof.

It is an additional object of the present invention to minimize the risk of injury or death of an individual resulting from harvesting stem cells from the brain of the individual.

It is a further object of the present invention to provide an efficient and economical way to harvest neural stem cells from an individual.

It is an additional object of the present invention to provide a method of treating a patient with a neurological condition by administering purified stem cells harvested from cerebrospinal fluid of the spinal cord of an individual.

It is a further object of the present invention to minimize the pain and suffering of a patient afflicted with a neurological condition by administering purified stem cells harvested from cerebrospinal fluid of the spinal cord of an individual.

It is an additional object of the present invention to provide a method of treating a patient through the use of purified neural stem cells in order to enhance recovery from a neurological condition.

It is a further object of the present invention to harvest neural stem cells from an individual with a minimally invasive procedure.

These and other aspects of the present invention will be more fully understood from the following detailed description of the invention and reference to the illustration appended hereto.

### BRIEF DESCRIPTION OF THE DRAWINGS

The FIGURE is a flow diagram illustrating the steps of the methods of the present invention.

### DESCRIPTION OF THE PREFERRED EMBODIMENTS

As used herein, “stem cells” mean cells capable of self-renewal, proliferation and differentiation into multiple different phenotypic lineages.

As used herein, “neural stem cells” mean stem cells that are self-renewing, multipotent cells which differentiate into nerve cells of the nervous system and shall expressly include, but not be limited to, neurons, astrocytes and oligodendrocytes.

As used herein, “individual” means a full-term human being having no recent history of an edematous brain condition.

As used herein, “patient” is meant to refer to mammalian members of the animal kingdom, including humans.

In an embodiment of the present invention, a method is provided for producing purified neural stem cells. As shown in the FIGURE, the method comprises harvesting fluid containing neural stem cells from cerebrospinal fluid (“CSF”) surrounding the spinal cord of an individual 1. The harvesting of the fluid is effected, for example, and without limitation, by intrathecal aspiration of the CSF contained in the annular region surrounding the spinal cord. The neural stem cells are isolated from the harvested fluid 2 using techniques well known in the art. The isolated neural stem cells are cultured 3 in a culture medium effective to induce proliferation of the neural stem cells. The cultured stem cells are purified 4 by, for example, immunocytochemical purification and other means known by those skilled in the art.

The regions of the spinal cord from which CSF is aspirated include the cervical region down to the sacral region and all regions in between. Preferably, CSF is aspirated from the fluid surrounding the lumbar region. Fluid from the CSF is collected in a syringe having a needle ranging in length of between 1 inch to 6 inches, preferably 3.5 inches. The gage of the needle can range from 10-27 gage, preferably 18-22 gage. Alternatively, CSF may be aspirated through an indwelling lumbar intrathecal catheter (gage) over a period of hours to days.

Imaging techniques, such as, for example, fluoroscopic imaging, are used to ensure that the needle is placed in the correct location of the annular region of the site of aspiration.

In a further embodiment, a method is provided for treating a patient afflicted with a neurological condition. The method comprises harvesting fluid containing neural stem cells from cerebrospinal fluid surrounding the spinal cord of an individual, isolating the neural stem cells from the fluid, culturing the neural stem cells in a culture medium effective to induce proliferation of the neural stem cells, purifying the cultured neural stem cells and administering the purified neural stem cells into a patient in need thereof. The neural stem cells harvested from an individual may be autologous administration to the same individual or may be heterologous administration to a patient other than the individual.

Administration of the purified neural stem cells results in the purified neural stem cells propagating in or adjacent to the site of the brain region afflicted with the neurological condition. For example, in the case of a stroke victim, the purified neural stem cells are administered in the region of the brain where the stroke occurred. In addition, the purified stem cells may be administered in a region remote from the site of the brain region afflicted with the neurological condition, such as a vein, as the neural stem cells are capable of delivering themselves to the afflicted site and seed themselves therein.

The amount aspirated from the CSF surrounding the spinal cord of the patient ranges from between about 5 ml to about 20 ml. In a preferred embodiment, a small amount of CSF (volume range 1-1000 cc) can be harvested. Although higher amounts of cerebrospinal fluid may be safely collected over a period of hours to days through an indwelling lumbar intrathecal catheter (draining CSF), no more than 10 cc of cerebrospinal fluid is suggested to be aspirated from the intrathecal space so that cerebral herniation is most unlikely. In our study, we have isolated neural stem and progenitor cells from as little as 1 cc.

Neurological conditions that can be treated according to the methods of the present invention include neurodegenerative or neurological diseases such as, for example, stroke, traumatic brain injury, traumatic spinal cord injury, Parkinson's disease, Alzheimer's disease, Huntington's disease, multiple sclerosis, or depression.

Techniques of purifying the neural stem cells are well known in the art and include, without limitation, immunocytochemical purification and other means known by those skilled in the art.

The following examples are intended to illustrate the invention and should not be construed as limiting the invention in any way.

### Example I

An example of the method of cell collection and processing will be considered.

The subjects are positioned in the lateral recumbent position with the knees tucked. Alternatively, subjects may be positioned in the sitting position. Fluoroscopic imaging, CT, or MRI may be used to identify the interspinous processes leading to intrathecal spaces. Identification of the interspinous processes may occur by any other method known in the art. The puncture site is first cleaned with 10% povidine-iodine solution followed by 2% chlorhexadine in 70% isopropyl alcohol. Skin prep procedure may vary. This is allowed to dry completely. Sterilization of the skin may occur by any technique known in the art. In a preferred aspect, a collection kit packaged in a sterile container can be used. In one particular embodiment, the collection kit can consist of (i) a sterile glass or plastic container with tight fitting cap, and (ii) a plastic, flexible, sealed collection bag in which the container is placed, and (iii) an identification label, which identified the source of the sample and time of collection. Sterilization of the containers can occur by any technique known in the art, including but not limited to, beta-irradiation, autoclaving of suitable materials in a steam sterilizer, etc. The collection may be shipped to the donor and placed in the surgical field in advance of the harvestation procedure. The cerebrospinal fluid is immediately transported on ice to a culturing facility in a sterile, closed container. Chilled sterile phosphate buffered saline (PBS, pH 7.4) with 0.6% glucose or DMEM/F12 may be added to the cerebrospinal fluid. If the cerebrospinal fluid sample requires shipping overnight to a culturing facility, the container is packed in ice (wet) and sent. All cerebrospinal fluid (“CSF”) samples were centrifuged between 600 g to 1000 g at room temperature for 6 to 10 minutes. The supernatant is discarded. Fresh growth medium is added to the pellet consisting of DMEM/HAMS F-12 (3:1), penicillin G, B27 (1:50; Gibco), human recombinant FGF-2 and EGF (both at 2Ong/ml; Sigma). The cellular solution is plated on substrate-free tissue culture flaks. Cultures are fed every other day by replacing approximately 50% of the media and adding growth factors to make the above concentrations.

Passaging is carried out every two weeks and consists of a gentle mechanical dissociation using a fine polished Pasteur pipette, after which the mixture of intact spheres and single cells are reseeded into fresh medium, as above but with N2 (1:100; Gibco) or B27 (1:50; Gibco). At varying points, a total cell count may be estimated using a 1 mL aliquot of spheres (taken from a 20 mL flask of cells which is shake randomly to distribute the spheres). Live cells may be counted using trypan blue exclusion to exclude dead cells.

Once a sufficient number of cells is achieved (typically 100,000 to 10,000,000 cells), the human neural stem cells described herein may be cryopreserved according to routine procedures. A controlled slow cooling rate is critical. Cells may be preserved in a freezing medium consisting of proliferating medium (absent the growth factors), 10% BSA (Sigma A3059), and 7.5% DMSO. Cells are centrifuged. Growth medium is aspirated and replaced with freeze medium. Cells are slowly frozen (giving a cooling rate of approximately 3 degree/hour typically over 12-24 hours) by placing in a container at −80° C. Following this, the specimen can be placed directly into liquid nitrogen (−196° C.) for permanent storage.

In order to identify cellular phenotypes during proliferation or differentiation of the neural stem cells, various cell surface or intracellular markers may be used.

### Example II

### Methods

Twenty healthy adult individuals are used in this study. CSF is obtained from the fluid surrounding the lumbar region of the spinal cord by lumbar puncture, a technique well known in the art. Approximately 10 ml of CSF per individual is aspirated. The fluid is collected in a syringe having a needle 3.5 inches. The gage of the needle is 18 gage.

Cell Culture

The aspirated fluid is placed in a flask and the flask is placed on a rotating orbital shaker for 25 minutes at 37° C. and 100 rpm. A single cell suspension results. The suspension is taken off from the flask and placed in a centrifuge tube with 3 mL of fetal bovine serum (FBS, Bioproducts) in order to inactivate the enzyme reaction. It then is washed in DMEM and centrifuged at 1500 rpm for 10 minutes. The wash then is suctioned off and the cells retrieved from the pellet are resuspended in 1×DMEM with 10% FBS and placed on ice. The DMEM wash is repeated as described above, all cells are re-suspended in a larger volume and then plated in a 100 mm Petri dish. Each plate is treated with 100 μL of Ampicillin in 100 mg/ml, 100 μL of bovine pituitary derived from fibroblast growth factor (FGF, Biomedical Technologies), 50 μL of Amphotericin (AMP, Mediatech) and 5 ng/mL of leukemia inhibitory factor (LIF; Sigma). Once the cells have attached, the cells are switched to serum-free conditions by placing them in neural basal media (Invitrogen) containing 10 ng/mL B27 (Invitrogen) supplement and 10 ng/mL epithelial growth factor (EGF). FGF is added separately to each plate at 10 ng/mL every 24 hours in order to avoid degradation as previously reported (Kanemura, Y. et al., Cell Transplant, 14:673-682, 2005).

Immunocytochemistry

Cells are purified in culture and tested by either plating a suspension of cells on to sterile gelatin-coated slides in a 100 mm Petri dish or into a cytospin (Shandon). The cells are fixed onto the slides using alcohol formaldehyde acetic acid (AFA) and rinsed with 1× phosphate buffered saline (PBS, 1:10, Mediatech). After removing the slides from the 1×PBS, a circle is etched in the slide using a diamond pencil. The slide is carefully dried with a paper towel avoiding the etched circle and placed immediately in a humidity chamber. A 5% milk (Carnation) block is added and kept within the etched circle and the humidity chamber sits for 1 hour at room temperature. When blocking is done, the slide is tapped on its side into an absorbent pad and any excess is wiped away around the fluid (some block remains on the fluid). A primary antibody is added and the slide is placed back into the humidity chamber and incubated overnight at 4° C. The primary antibodies that are used are: GFAP (Chemicon), OSP (Abcam) PSA-NCAM (Chemicon) and Nestin (Abcam). After the primary incubation, slides are tapped on their sides to remove any excess and then are rinsed with PBS. The slides are placed in a glass slide holder and are rotated on a red rotor for two five-minute washes. The PBS is discarded, refilled and the slides are rotated for an additional ten minutes. The PBS is tapped off of the slides, the slides are dried and then placed back in the humidity chamber. One to two drops of biotinylated anti-mouse or anti-rabbit immunoglobin is placed on the fluid and the chamber is covered and incubated at room temperature for 30 minutes. Slides are washed as before and placed back into the chamber, 1 to 2 drops of streptavidin alkaline phosphatase conjugate is added to the fluid and they are again incubated at room temperature for 30 minutes. This is rinsed off with two five minute washes in a glass chamber filled with PBS. Fast red napthol substrate containing 125 mM levamisole (Vector) to block endogenous phosphatase is added and left on the slides for five minutes. Staining intensity is checked and ceased by rinsing the slides with PBS and placing them in the glass chamber containing PBS. Counterstaining can be done for nuclei using Mayer's hemotoxylin. The slides are preserved utilizing Dako Glycergel mounting media.

Analysis

The presence of neuronal progenitor stem cells from the CSF surrounding the spinal cord of donors will be determined by immunocytochemistry by the positive staining of neurons with Nestin and PSA-NCAM, the positive staining of oligodendrocytes with OSP and the positive staining of astrocytes or glial cells with GFAP.

If neurospheres develop from the donor individual's sample, the diameter of the neurosphere is measured daily using a micrometer. These measurements are analyzed to show that the neurospheres can proliferate in colonies. A growth curve is created on disassociated neurospheres isolated from the plate from which they developed.

The purified neural stem cells are preferably stored cryogenically under conditions well known in the art which are similar to cord blood banks. They may be thawed immediately prior to administration to an individual.

Neural Stem Cell Transplantation

Transplantation is performed according to standard techniques well known in the art. Specifically, approximately 500,000 neural stem cells are transplanted into the brain of a patient suffering from stroke in which the patient is positioned in a stererotaxic instrument. The region of transplantation is in or adjacent to the site where the stroke injury occurred. A midline incision is made in the scalp and a hole drilled for the injection of cells. The cells are injected using a glass capillary attached to a 10 μl Hamilton syringe. Following implantation, the skin is sutured closed.

Whereas particular embodiments of this invention have been described above for purposes of illustration, it will be evident to those skilled in the art that numerous variations of the details of the present invention may be made without departing from the invention as defined in the appended claims.

What is claimed is:

1. A method of producing purified neural stem cells, comprising:
harvesting fluid containing neural stem cells from cerebrospinal fluid surrounding the spinal cord or spinal nerve roots of an individual;
isolating neural stem cells from the fluid;
culturing said isolated neural stem cells in a culture medium effective to induce proliferation of said neural stem cells; and
purifying said cultured neural stem cells.

2. The method according to claim 1, wherein the fluid is harvested from the cerebrospinal fluid of the individual by intrathecal aspiration.

3. The method according to claim 2, wherein said intrathecal aspiration is effected by a syringe.

4. The method according to claim 1, wherein said harvesting of said fluid is aspirated from the cerebrospinal fluid surrounding a region of the spinal cord or spinal nerve roots which includes the cervical region to the sacral region of the spinal cord and all regions in between.

5. The method according to claim 1, wherein said harvesting of said fluid is aspirated from the cerebrospinal fluid surrounding the lumbar region of the spinal cord.

6. The method according to claim 1, wherein said purification is effected by immunocytochemical purification.

7. The method according to claim 1, wherein said culturing is effected in vitro.

8. The method according to claim 1, wherein said culturing producers clinically relevant quantities of said neural stem cells.

9. A method of treating a patient afflicted with a neurological condition, comprising:
harvesting fluid containing neural stem cells from the cerebrospinal fluid surrounding the spinal cord or spinal nerve roots of an individual;
isolating neural stem cells from said fluid;
culturing said isolated neural stem cells in a culture medium containing growth factors effective to induce proliferation of said neural stem cells;
purifying said cultured neural stem cells; and
administering said purified neural stem cells into the patient.

10. The method according to claim 9, wherein said culturing is effected in vitro.

11. The method according to claim 9, including administering said purified neural stem cells in a therapeutically effective amount.

12. The method according to claim 9, including after said purifying and prior to said administering storing cord neural stem cells.

13. The method according to claim 12, including employing cryopreservation to store said purified neural stem cells.

14. The method according to claim 13, including thawing said cryopreserved stem cells prior to administration.

15. The method according to claim 9, wherein about 5 ml to 20 ml of purified neural stem cells are administered to said patient.

16. The method according to claim 9, wherein said individual is a full-term infant or older who recently has not experienced an edematous brain condition.

17. The method according to claim 9, wherein said patient is a human being.

18. The method according to claim 9, wherein said neurological condition is selected from the group consisting of stroke, traumatic brain injury, traumatic spinal cord injury, Parkinson's disease, Alzheimer's disease, Huntington's disease, multiple sclerosis and depression.

19. The method according to claim 9, wherein said patient is a stroke victim.

20. The method according to claim 9, wherein said purified stem cells are administered to the stroke patient in a brain region where the stroke occurred.

21. The method according to claim 9, wherein said purified stem cells are administered to the stroke patient in a brain region adjacent to the brain region where the stroke occurred.

22. The method according to claim 9, wherein said purified stem cells are administered to the stroke patient in a region remote from the brain region where the stroke occurred.

23. The method according to claim 9, wherein said patient is said individual.

24. The method according to claim 9, wherein said patient is not said individual.

25. The method according to claim 9, wherein said administration results in said purified neural stem cells propagating in the region where the stroke occurred.
